(19) United States
(12) Patent Application Publication
(10) Pub. No.: US 2007/0224150 A1
(43) Pub. Date: Sep. 27, 2007

(54) Growth factor for hair and skin treatment

(76) Inventor: Yongji Chung, (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/385,200

(22) Filed: Mar. 21, 2006

Related U.S. Application Data
(60) Provisional application No. 60/664,772, filed on Mar. 24, 2005.

(51) Int. Cl. A61K 38/18; A61K 8/64

(52) U.S. Cl. 424/70.14; 424/401; 977/926; 514/12

(57) ABSTRACT

The present invention relates to a method of treatment for slowing the progress of skin aging comprising contacting the skin with an amount effective to slow skin aging of a composition comprising one or more compounds selected from the group consisting of EGF, bFGF, IGF-1, KGF, TGF-β3, TRX, VEGF, TRX, aFGF, FGF-10, copper peptide, acetyl hexapeptide, palmitoyl pentapeptide, CPP, and UDN glycoprotein.

### CROSS-REFERENCE TO RELATED APPLICATIONS

The present application claims priority to U.S. Provisional Patent Application No. 60/664,772 filed on Mar. 23, 2005 and incorporated herein by reference.

### STATEMENT REGARDING FEDERALLY SPONSORED RESEARCH OR DEVELOPMENT

Not Applicable.

### REFERENCE TO A SEQUENCE LISTING

Not Applicable.

### BACKGROUND OF THE INVENTION

It is known that the aging process decreases cytokine production in the human body. Decrease in cytokine activity can induce wrinkles, hair-loss, excess fat, dermatitis, and other aging-related conditions. In addition, it is known that changes in cytokine activities are closely correlated with certain diseases. Various attempts to use cytokines to treat these diseases have been and are being actively pursued.

Features of aging skin may include: thinning of the epidermis and dermis; coarsening of the skin texture, including enlargement of pores; laxity with wrinkling; discoloration, including yellowing, bronzing, and brown spots; and telangiectasia, or “broken veins”. Restoration of aged skin tissue or chapped skin tissue to its original condition is closely associated with cytokine activity. Cytokines may be administered to patients orally or by parenteral administration, such as by injection or endermic application. However, these methods require continuous administration of a large amount of expensive cytokines to the patient until complete recovery is achieved. These methods can be therefore costly and time-consuming.

Alternatively, a substance that promotes the production of cytokines may be administered. Like direct administration of cytokines, the cytokine production enhancer substance may be administered to the patient orally or by parenteral administration such as injection or endermic application. However, like the administration of cytokines, the administration of cytokine production enhancers can be costly and time-consuming.

The administration of large amounts of cytokines for extended periods of time can be further problematic in that the treatment often disrupts the patient's overall metabolism. In addition, external application of cytokines are ineffective in achieving a local effect, due to decomposition or poor absorbability of the compound. Therefore, a safer method for treating aged or chapped skin and promote hair growth is desirable.

### BRIEF SUMMARY OF THE INVENTION

In a first set of representative embodiments, the present invention provides a method of treatment for slowing the progress of skin aging comprising contacting the skin with an amount effective to slow skin aging of a composition comprising one or more compounds selected from the group consisting of EGF, bFGF, IGF-1, KGF, TGF-β3, TRX-1, VEGF, aFGF, FGF-10, copper peptide, acetyl hexapeptide, palmitoyl pentapeptide, CPP, and UDN glycoprotein.

In a second set of representative embodiments, the present invention provides a composition for slowing the progress of skin aging, the composition comprising one or more compounds selected from the group consisting of EGF, bFGF, IGF-1, KGF, TGF-β3, TRX, VEGF, aFGF, FGF-10, copper peptide, acetyl hexapeptide, palmitoyl pentapeptide, CPP, and UDN glycoprotein.

In a third set of representative embodiments, the present invention provides a method of treating hair loss comprising contacting the skin with an amount effective to treat hair loss of a composition comprising one or more compounds selected from the group consisting of bFGF, KGF, IGF-1, SCF, VEGF, copper peptide, aFGF, Noggin and thymosinβ4.

In a fourth set of representative embodiments, the present invention provides a composition for treating hair loss, the composition comprising one or more compounds selected from the group consisting of bFGF, IGF-1, and VEGF.

In a fifth set of representative embodiments, the present invention provides a method for treating acne comprising contacting the skin with a composition comprising one or more compounds selected from the group consisting of EGF, bFGF, TRX, UDN glycoprotein, IL-4, IL-10 and SCF.

In a sixth set of representative embodiments, the present invention provides a composition for treating acne, the composition comprising one or more compounds selected from the group consisting of EGF, bFGF, TRX, UDN glycoprotein, IL-4, IL-10 and SCF.

In a seventh set of representative embodiments, the present invention provides a method for treating acne comprising contacting the skin with a composition comprising one or more compounds selected from the group consisting of EGF, IGF-1, bFGF, copper peptide and UDN glycoprotein.

In an eighth set of representative embodiments, the present invention provides a composition for treating acne, the composition comprising one or more compounds selected from the group consisting of EGF, IGF-1, bFGF, copper peptide and UDN glycoprotein.

In a ninth set of representative embodiments, the present invention provides a method for treating atopic dermatitis comprising contacting the skin with a composition comprising one or more compounds selected from the group consisting of EGF, bFGF, TRX, UDN glycoprotein, IL-4, IL-10 and SCF.

In a tenth set of representative embodiments, the present invention provides a composition for treating atopic dermatitis, the composition comprising one or more compounds selected from the group consisting of EGF, bFGF, TRX, UDN glycoprotein, IL-4, IL-10 and SCF.

In an eleventh set of representative embodiments, the present invention provides a method for treating psoriasis comprising contacting the skin with an amount effective to treat psoriasis of a composition comprising one or more compounds selected from the group consisting of EGF, bFGF, TRX, UDN glycoprotein, IL-4, IL-10 and SCF.

In a twelfth set of representative embodiments, the present invention provides a composition for treating psoriasis, the composition comprising one or more compounds selected from the group consisting of EGF, bFGF, TRX, UDN glycoprotein, IL-4, IL-10 and SCF.

In a thirteenth set of representative embodiments, the present invention provides a method for increasing lipogenesis comprising administering to a patient an amount effective to increase lipolysis of a composition comprising IGF-1, and L-carnitine.

In a fourteenth set of representative embodiments, the present invention provides a composition for increasing lipogenesis, the composition comprising IGF-1, and L-carnitine.

In a fifteenth set of representative embodiments, the present invention provides a method for treating leukoplakia comprising contacting the skin with an amount effective to treat leukoplakia of a composition comprising one or more compounds selected from the group consisting of bFGF and SCF.

In a sixteenth set of representative embodiments, the present invention provides a composition for treating leukoplakia, the composition comprising one or more compounds selected from the group consisting of bFGF and SCF.

In a seventeenth set of representative embodiments, the present invention provides a method for reducing sun exposure of the skin comprising applying to the skin a composition comprising one or more compounds selected from the group consisting of EGF, bFGF, IGF-1, TRX, CPP, and UDN glycoprotein.

In an eighteenth set of representative embodiments, the present invention provides a composition for reducing sun exposure of the skin, the composition comprising one or more compounds selected from the group consisting of EGF, bFGF, IGF-1, TRX, CPP, and UDN glycoprotein.

In a nineteenth set of representative embodiments, the present invention provides a method for treating atopic dermatitis comprising contacting the skin with an amount effective to treat atopic dermatitis of a composition comprising one or more compounds selected from the group consisting of IL-10, TRX, EGF and bFGF.

In a twentieth set of representative embodiments, the present invention provides a composition for treating atopic dermatitis, the composition comprising one or more compounds selected from the group consisting of IL-10, RRX, EGF and bFGF.

These and other features of the present teachings are set forth herein.

### BRIEF DESCRIPTION OF THE FIGURES

The skilled artisan will understand that the figures, described below, are for illustration purposes only. The drawings are not intended to limit the scope of the invention in any way.

FIG. 1 illustrates the proliferation percentages of EGF-treated cells.

FIG. 2 illustrates the proliferation percentages of AC-treated cells.

FIG. 3 illustrates a comparison between EGF-treated cells, AC-treated cells and untreated control cells.

### DETAILED DESCRIPTION

Cytokines as referred to herein include epidermal growth factor (hereinafter referred to as EGF), insulin-like growth factor (hereinafter referred to as IGF), basic fibroblast growth factor (hereinafter referred to as bFGF), thioredoxin-1 (hereinafter referred to as TRX-1), keratinocyte growth factor (hereinafter referred to as KGF), stem cell factor (hereinafter referred to as SCF), transforming growth factor beta 3 (hereinafter referred to as TGF-β3), interleukin-10 (hereinafter referred to as IL-10), platelet-derived growth factor (hereinafter referred to as PDGF), vascular endothelial cell growth factor (hereinafter referred to as VEGF), acidic fibroblast growth factor (hereinafter referred to as aFGF), fibroblast growth factor 10 (hereinafter referred to as FGF-10), interleukin-4 (hereinafter referred to as IL-4), copper peptide, Noggin, thymosin β4, hepatocyte growth factor (hereinafter referred to as HGF), acetyl hexapeptide, palmitoyl pentapeptide, calcium pyrphosphate (hereinafer referred to as CPP), Ulmus Davidana Nakai glycoprotein (hereinafter refered to as UDN glycoprotein).

Of these cytokines, those which activate tyrosine or serine/threonine kinases, and also activate the phosphorylation of tyrosine, or serine and/or threonine residues on subunits of tyrosine or serine/threonine kinase-linked receptors, respectively, are preferred effectors.

EGF is composed of 53 amino acids and is found in varying concentrations in milk, saliva, urine, plasma, and also in most other body fluid. Mitogen activity in EGF provides a signaling mechanism to the damaged and aged epidermis cells to stimulate mitosis, cell division, or regeneration. EGF promotes cell growth and differentiation, is essential in embryogenesis, and plays an important role in wound healing. EGF expedites the cell proliferation of fibroblast cell which synthesize collagen and elastin which help skin reproduce collagen and elastin naturally from within. The effects of application of EGC on skin includes, but is not limited to, increasing skin elasticity, promoting hair growth, promoting wound healing, promoting anti-aging conditions and treatment of other body aliments.

IGF-1 is 7.6 kDa mono chain polypeptide hormone structurally similar to pro-insulin, which is synthesized at the liver, fibroblast cell. It regulates cell growth and expedites development, especially in the nerve cells, as well as cellular DNA synthesis. IGF-1 has been used as an indicator of the aging process in the skin layer where a drop in IGF-1 level has been directly related to the aging of the skin. Introduction of IGF-1 into the skin layer will stimulate and expedite the biosynthesis of hyaluronic acid and collagen, naturally returning the skin to a healthier and younger form. IGF-1 is secreted as a result of GH stimulation and other hormones which act to increase IGF-1 synthesis and it also enhances the normal resisting action of insulin. The effects of application of IGF-1 on skin includes, but is not limited to, increasing skin elasticity, promoting hair growth, promoting wound healing, promoting anti-aging conditions and treatment of other body aliments. bFGF, also called FGF-2 or heparin-binding growth factor 2 (hbgf-2) or prostratropin, is an angiogenic agent with gene therapy uses such as atherosclerosis therapy. bFGF is an essential constituent for restoration of the aging tissue and treatment of wounds and bruises. It promotes wound healing and revascularization of tissues. Along with EGF and other growth factors or in combination thereof, bFGF improves the elasticity of the skin and smooth out the wrinkles with increasing biosynthesis of natural collagen and Elastin, which are the main structural elements of a healthy skin layer. bFGF also expedites the cell multiplication and division of the dermis extra-cellular matrix fibroblast, keratinocytes and the biosynthesis of hyaluronic acid by interacting with EGF and IGF-1. bFGF in combination with at least EGF and IGF-1 generates biosynthesis of cytokines. The bFGF, EFG, and IGF-1 combination expedites the hair growing cycle by increasing synthesis of collagen and Elastin by the root of the hair, keeping them healthy and preventing the hair loss cycle. bFGF further slows the progression of hair from undergoing grey de-colorization . The effects of application of bFGF on skin includes, but is not limited to, increasing skin elasticity, promoting hair growth, promoting wound healing, promoting anti-aging conditions and treatment of other body aliments.

TRX is a strong anti-oxidant that interacts with free radicals and prevents the free radicals from contributing to the aging process in the skin layer and halts cells from perishing from the influence of the free radicals. TRX protects the skin layer from aging by affecting cells' growth via controlling DNA factors which bind transcriptional factors. The effects of application of TRX on skin includes, but is not limited to, acting as sunblock, increasing skin elasticity, promoting hair growth, promoting wound healing, promoting anti-aging conditions and treatment of other body aliments.

KGF was first described in 1989 as a human growth factor that stimulated the epithelial cell proliferation and provides protection against a wide variety of injurious stimuli. KGF is in the family of super fibroblast growth factor, which influences proliferation and multiplication of various cell types. KGF provides adhesive affects between the cells, spreading and proliferation between cells, and has key functions in cell healing from various forms of damages including aging. KGF is a key component in the early cycle of hair growth and works as a bridge between each cell's generation cycle. The effects of application of KGF on skin includes, but is not limited to, acting as sunblock, increasing skin elasticity, promoting hair growth, promoting wound healing, promoting anti-aging conditions and treatment of other body aliments.

SCF is composed of 164 amino acids and is produced in both a soluble and a membrane bound form SCF expression also has been detected in the human keratinocytes in the skin, and along the migratory routes of melanocytes allowing the cell to home in onto the proper development sites. SCF and its receptor c-kit are important factors for melanocyte survival during development, and mutations in the genes result in un-pigmented hair. The effects of application of SCF on skin includes, but is not limited to, increasing skin elasticity and melancyte production and treatment of other body aliments.

TGF is one of the several proteins secreted by transforming cells that can stimulate the growth of all normal cells. TGF generates a powerful multifunctional cytokine that affects the growth and differentiation of skin cells to keep the skin younger and healthier. For example by converting and repairing old and damaged cells into healthier and more productive cell. TGF prevents scar tissue from forming and heals already damaged cells. It increases synthesis of matrix proteins by binding itself to EGF receptor and stimulating the growth of endothedial cells. The effects of application of TGF on skin includes, but is not limited to, increasing skin elasticity, promoting hair growth, promoting wound healing, promoting anti-aging conditions and treatment of other body aliments.

Human IL-10 or Human oligopeptide-8 has a length of 16 a.a. and a molecular weight of 18.6 kDa. IL-10 regulates the immune response through autocrine signaling and paracrine signaling. It serves as a biological inducer of immune tolerance through suppression of T helper cells. The effects of application of IL-10 on skin includes, but is not limited to, relieving skin inflammation, increasing wound healing, improving atopic dermatitis and psoriasis and treatment of other body aliments.

PDGF is also known as human oligopeptide-10 and has a length of 125 a.a. and molecular weight of 14.3 kDa. PGDF helps with wound repair by facilitating blood vessel formation at a wound area and promoting secretion of other wound repair related growth factors PDGF also promotes skin regeneration by facilitating the proliferation of the fibroblast cells that synthesize collagens. The effects of application of PDGF on skin includes, but is not limited to, promotion of anti-aging conditions, increasing hair growth, preserving skin elasticity and treatment of other body aliments.

VEGF is a diffusible and specifically required for endothelial cells, which suggests that this cell molecule may play a unique role in the regulation of angiogenesis. VEGF plays an important role in the control of perifollicular vascularization during the hair growth cycle. The effects of application of VEGF includes, but is not limited to, promotion of anti-aging conditions, increasing hair growth, preserving skin elasticity and treatment of other body aliments.

aFGF serves as a modifier of endothelial cell migration and proliferation and thus may be important in neovascularization. It has potent mitogen activity in vitro for many cells of ectodermal and mesodermal embryonic origin including skin-derived epidermal keratinocytes, dermal fibroblasts and vascular endothelial cells and it accelerates wound healing. The effects of application of aFGF on skin includes, but is not limited to, promotion of anti-aging conditions, increasing hair growth, preserving skin elasticity and treatment of other body aliments.

FGF-10 also accelerates wound healing by inducing proliferation and differentiation of human keratinocytes. FGF-10 functions similairly to FGF-7. The effects of application of FGF-10 on skin includes, but is not limited to, promotion of anti-aging conditions, increasing hair growth, preserving skin elasticity and treatment of other body aliments.

IL-4 promotes the proliferation and differentiation of activated B-cells. IL-4 has a synergistic effect with EPO and G-CSF/Epo in the generation of colonies containing granulocytes or erythroid progenitor cells to induce proliferation in normal human keratinocytes. The application of IL-4 includes, but is not limited to, improvement of psoriasis and other body aliments.

Copper peptide increases the activation of metalloproteinases of the skin. It also improves the elasticity of the skin and smoothes out wrinkles with increasing biosynthesis of collagen and elastin. Copper peptide slows the progression of the aging process with the activation of the skin function by generating the proteoglycans and glycosaminoglycans which are main structural elements of epidermis. Copper peptide also slows the aging of the skin through the activation of superoxide dismutase which impacts the metabolism of skin cell. Copper peptide promotes hair growth by increasing the size of hair follicles and stops the hair from falling out. The effects of application of copper peptide to skin includes, but is not limited to, promotion of anti-aging conditions, improvement of skin elasticity, prevention of hair-loss, thickening of hair, promotion of wound healing and treatment of other body aliments.

Noggin neutralizes the inhibitory activity of BMP2/4 by the BMP antagonist. Noggin is also essential for HF (Hair Follicle) induction. BMP2/4 serves as an important inhibitor of anagen initiation in postnatal skin and the neutralization of BMP4 by its antagonist noggin is required for the HF telogen-anagen transition. The effects of application of noggin on skin includes, but is not limited to, promotion of hair growth and treatment of other body aliments.

Thymosin was originally isolated from calf thymus and designated thymosin beta 4(1981) It affects the follicle stem cell growth, migration, differentiation, and protease production. Thymosin β4 increases hair growth by activation of hair follicle stem cells and it acts to promote hair growth. The effects of application of thymosin on skin includes, but is not limited to, promotion of hair growth and treatment of other body aliments.

hGH decreases body fat and increases muscle strength and the immune system. hGH has been found to reduce stress levels. The effects of application of hGH on skin includes, but is not limited to, promotion of hair growth, improvement of skin elasticity, deduction of fat cells and treatment of other body aliments.

Acetyl hexapeptide is a Botox-like hexapeptide, which is a non-toxin substitute material. Acetyl hexapeptide is advantageous because it has none of the known side effects, such as severe allergy, which existing Botox ingredients can cause. Acetyl hexapeptide serves as an anti-wrinkle and anti-aging ingredient while not having a toxic influence on the human body and environment. Acetyl hexapeptide also is easy to percolate through the skin because it is a low molecular peptide and then it directly acts to muscle tissue. This process increases the skin elasticity in a short time. This prevents aging by influencing secretion of catecholamines (adrenalin and noradrenalin) which are main causes of wrinkle formation. Acetyl Hexa-peptide controls the formation and stabilization of SNARE complex. SNARE complex, which is formed by Acetyl Hexa-peptide, directly acts on muscles, then activates them and consequently affects wrinkle improvement. The effects of application of acetyl hexapeptide on skin includes, but is not limited to, promotion of anti-aging conditions, improvement of skin elasticity and treatment of other body aliments.

Palmitoyl pentapeptide hastens the activation of skin cell with stimulation of the generation of collagen and glycosaminoglycan without side effects. Palmitoyl pentapeptide restores UV damaged skin and improves the fine texture of the skin, while reducing the volume and depth of wrinkles. It generally has approximately twice the effect of retinoid or vitamin A on wrinkle improvement without the potential side effects and irritation associated with retinoid. It is more stable and easier to store in normal condition. The effects of application of palmitoyl pentapeptide on skin includes, but is not limited to, promotion of anti-aging conditions and improvement of skin elasticity.

UDN glycoprotein is the enriched glycoprotein isolated from Ulmus davidiana Nakai. Ulmus Davidiana Nakai is a deciduous tree that inhabit in Korea and the like. Glycoprotein from UDN was shown to have strong scavenging activities against oxygen free radicals as detected by different oxygen-radical formation assays.

The combination of above-mentioned cytokine and growth factors is applicable, but is not limited to, anti-aging and wrinkle-defense products, anti-hair loss products, fat burning products, anti-acne products, mesotherapy products, sun-block products, anti-atopic dermatitis products and anti-psoriasis products.

For skin care, such as anti-aging and anti-wrinkle products, the combination of ingredients includes, but is not limited to, EGF, bFGF, IGF-1, KGF, TGF-β3, TRX-1, VEGF, aFGF, FGF-10, copper peptide, acetyl hexapeptide, palmitoyl pentapeptide CPP, UDN glycoprotein and herbal extracts. EGF and IGF-1 promote epidermis cell growth and bFGF promotes dermis cell growth and collagen synthesis. Functions of cytokine and growth factors promote epidermal tissue regeneration band stimulate keratinocyte and fibroblast differentiation causing the reduction of reactive oxygen species (ROS).

The beneficial effect of exogenous growth factors in treatment of wound repair as well as the identification of the in vitro activities of many growth factors and cytokines have implicated these proteins as key regulators of the wound healing process.

The mechanism of skin regeneration is according the following stages. In the epidermis EGF and IGF-1 promote epidermis cell proliferation. This activity may provoke expression of other cytokines which affect skin cell survival and proliferation. bFGF is then added which not only increase synthesis of collagen but also upregulates EGF activity. bFGF also increases collagen synthesis that makes tight collagen lattice in the dermis which provides skin elasticity. IGF-1 is also added in the epidermis and plays a role similar to that of human growth factor (HGH). IGF-1 is a key protein in the formation of blood vessel, bones, muscles, and neurons. By introducing IGF-1, this provides new blood vessels in physically damaged and old tissues which have decreased blood vessels. Therefore, new vessel formations stimulate cell proliferation with delivery of nutrients and removal of CO₂ and hazardous cell wastes from cells.

For hair growth and prevention of hair loss products, the combination of ingredients includes, but is not limited to, bFGF, KGF, IGF-1, SCF, VEGF, copper peptide, aFGF, noggin, thymosin β4, and herbal extracts.

The herbal extracts include, but are not limited to, ginkgo extracts, glycyrrhiza extracts, mantididootheca extracts, mulberry root bark extracts, polygonium multiflorum extracts, thujae orientalis extracts, VF-1 ( a flavonoid) and UDN glycoprotein.

The hair follicle periodically synthesizes biologic fibers commonly referred to as hair. Every hair follicle undergoes cyclic growth from an active phase (anagen) through a regression phase (catagen) and resting phase (telogen). Although the precise mechanism is not known it is believed that regulating molecules expressed in epithelial (matrix) cells under the influence of mesenchymal (papilla) cells may have a crucial role in the terminal differentiation of follicular matrix cells. Recently, particular families of growth factors have been reported to be involved in the regulation of hair morphogenesis and cyclic hair growth. They are polypeptides or proteins produced by the cells and function as cell growth regulatory molecules. For their hydrophylic properties, growth factors mediate their signals by binding to their specific receptors located on the cell surface. Hair follicle growth has been found to be inhibited by EGF, EGF-2 and TGF-beta while such growth was simulated by the administration of paracrine growth factors such KGF(FGF-7), IGF-1 and HGF.

The advantage of the embodiments of the present invention for treatment of skin and hair includes, but is not limited to normalization of the hair growth cycle, increasing hair growth, prevention of hair loss, synthesis of collagen and elastin, supplement of alimentation in the hair follicle, encouraging hair growth by increasing the size of hair follicle and preventing or inhibiting the hair from falling out

For aspects of the invention that are directed to body care products, such as fat-burning, the combination of ingredients includes, but is not limited to, EGF, bFGF, IGF-1, KGF, TGF, TRX, VEGF, copper peptide, acetyl hexapeptide and palmitoyl pentapeptide.

IGF-1 is a growth hormone and helps burn fat to increase muscle. GH promotes body growth via its indirect effect of stimulation IGF-1 production. IGF-1 is produced by liver and other tissues in response to GH binding. After entering blood stream, IGF-1 targets on its receptor in skeletal muscle, bones and cartilage. It stimulates protein synthesis and cell division.

The growth factors and cytokines induce lipolysis efficiency through blood circulation improvement which causes fat reduction by use with a slimming massage program. This in turn will increase basal metabolism amount and inhibit the function of phosphodiesterases such as caffeine. There is a B-receptor that helps to break down fat faster in our body. This functions to promote lipolysis by increasing cyclic-AMP.

Hormones react directly on fatty cells through adrenoreceptor by dissolving fat. These hormones include, but are not limited to, AR growth hormones, thyroid hormones, glucagons, and many other hormones. Adrenoreceptors are α-AR and β-AR. Beta-AR is also used when the body is dissolving fat.

Fat burning complex, such as, L-carnitine, IGF-1 and caffeine are capable of burning large amounts of fat when used with proper cardio exercise. The fat burning complex will decrease body fat by burning fat, increase energy use, and basic body metabolism. It also prevents dissipation of NE (Nonrepinenphrine) which increases body heat, increase cAMP to burn body fat.

The fat burning complex contemplated as an aspect of this invention regulates the formation of acetyl-CoA-carboxylase, which is a major hormone involved in forming of body fat. It decreases formation of cholesterol by regulating squalene epoxidase, (SE), which helps to form cholesterol. Over consumption of food builds up glycogen and increases body fat. By utilizing glycogen, the body burns fat and calories and also increases blood circulation, which helps the body burn fatty tissues effectively. Ginkgo extract has lipolysis efficiency through blood circulation improvement. Fast and more efficient results can be reached by burning fat and building muscle to increase elasticity, which helps to burn fat as well as prevent fat build up.

For products for the skin condition and diseases such as acne, atopic dermatitis, and psoriasis, the ingredients include, but are not limited to, EGF, bFGF, TRX-1, UDN glycoprotein, IL-4, IL-10, SCF, copper peptide, and anti-acne complex. Specifically, for anti-acne products, the ingredients include, but are not limited to, EGF, bFGF, TRX, copper peptide and UDN glycoprotein. For anti-proriasis, the ingredients include, but are not limited to, IL-10 and UDN glycoprotein. For anti-atopic dermatitis, the ingredients include, but are not limited to, IL-10, TRX, EGF and bFGF. For anti-leukoplakia products, the ingredients include, but are not limited to, bFGF and SCF.

For anti-acne products contemplated at embodiments of this invention, the preferred process is a three-step system. The first step is an anti-bacterial phase which includes ingredients IgY and nisin. The second step is an anti-inflammatory phase which includes UDN glycoprotein. This phase includes CG-Anti-acne complex specific for plural bacteria which has effects useful toward prevention of bacterial disease. The third step is a repair and remodeling phase which includes EGF, bFGF and copper peptide. The third phase promotes epidermal cell proliferation collagen and elastin synthesis to repair skin barrier protection for better skin formation. The anti-acne complex includes acne antibody which are anti-Propionibacterium acnes, anti-Staphylococcus epidermidis, anti-enterotoxigenic E. coli (ETEC).

Ingredients for anti-aging mesotherapy embodiments of the invention include, but are not limited to, cytokines: EGF, IGF-1, bFGF, copper peptide, vitamins: B complex, C, H, A, D, E, K, minerals: Ca, Mg, K, Na, amino acids: alanine, arginine, glutamine, lysine, nucleic acids: adenosine cyclic phosphate, cytosine, guanosine, tymine; 7. coenzymes: CoA, Cocarboxylase, NAD, FAD, and reducing agent: gluthation.

Ingredients for Anti-Hair Loss Mesotherapy embodiments of the invention include, but are not limited to, cytokines: IGF-1, bFGF, VEGF; peptide: copper peptide; vitamins: B complex, C, H, A, D, E, K; minerals: Ca, Mg, K, Na; amino acids: alanine, arginine, glutamine, Lysine; nucleic acids: adenosine cyclic phosphate, cytosine, guanosine, thymine; coenzymes: CoA, Cocarboxylase, NAD, FAD and reducing agent: gluthation.

Ingredients for sun block embodiments of the invention include, but are not limited to EGF, bFGF, IGF-1, TRX, CPP, UDN glycoprotein, herbal extracts, octyl methoxycinnamate, titanium dioxide, zinc oxide, octyl saliclate.

Ingredients for anti-atopic dermatitis embodiments of the invention include, but are not limited to IL-10, TRX, EGF, bFGF. This is to obtain Th1/Th2 cytokine balance which will inhibit IL-5 production by resting T cells and by TH0 and TH2 clones. There is down regulation of the expression of co-stimulatory molecules on APC. The epidermal cell growth occurs by upregulation of cell division. Collagen and Elastin synthesis is encouraged by increasing fibroblast cells which causes the regular immune condition to balance TH1 and prevent atopic dermatitis outbreak.

Ingredients for anti-psoriasis embodiments of the invention include, but are not limited to IL10, and UDN glycoprotein. This will achieve immunotherapy balance between Th1- and Th2- type cytokines and blocks interferon-gamma synthesis in Th1 cells which will reduce both MHC expression and production of pro-inflammatory cytokines in monocytes and macrophages

In any embodiment it is contemplated that it may be formulated as a lotion, a cream, or a power for topical application. Typical lotions can be formulated with an aqueous or oily base, and can include stabilizing agents, emulsifying agents, dispersing agents, suspending agents, thickening agents, coloring agents and the like.

Powders can be formulated with a suitable powder base, such as talc, lactose, starch and the like. Ointments, pastes, creams and gels can contain suitable excipients, such as paraffins, starch, tragacanth, cellulose derivatives, polyethylene glycols, silicones, talc and zinc oxide. Compositions can be combined with a standard base that may include emollients, lubricants, emulsifying agents, thickening agents, humectants, preservatives, antifungal agents, fragrances and wetting agents.

In a set of embodiments of the invention, cytokines and growth factors can be incorporated into a cream or lotion as microspheres, as is well known in the art.

In another set of embodiments, it is contemplated that the cytokines and growth factors can be incorporated into a cream or lotion as nanospheres, as is well known in the art. Examples of incorporating nanospheres into skin creams or lotions are disclosed in U.S. Pat. No.: 5,554,374 and U.S. Pat. No.: 6,203,802.

Topical pharmaceutical formulations may include one or more preservatives or bacteriostatic agents, such as methyl hydroxybenzoate, propyl hydroxybenzoate and benzalkonium chloride.

The compositions of the present invention can also include, for example, vehicles including, but not limited to, water or alcohol; humectants, including, but not limited to, glycerin; buffering agents including, but not limited to, citric acid and sodium citrate; viscosity adjusters, including, but not limited to, carbomer gelling agents, gum derivatives, and the like; preservatives including, but not limited to, methylparaben, propylparaben, and phenoxyethanol; emulsifiers including, but not limited to, polysorbitate 80, glyceryl distearate, POE 10 stearyl ether, ceateareth 20 and stearyl alcohol, and ceteareth 20 and cetearyl alcohol; conditioning agents including, but not limited to, octyl hydroxystearate; emollients including, but not limited to, cholesterol NF, petrolatum, mineral oils and esters including, but not limited to, isopropyl myristate, isopropyl palmitate, 1-decene polymer (hydrogenated), and C₁₂ -C₁₅ alcohol benzoates; thickness, including, but not limited to, polyacrylamide, C₁₃ -C₁₄ isoparafin, and laureth-7; antioxidants, including, but not limited to ascorbic acid, (BHT), tocopheryl acetate, and the like; UV stabilizers; UV radiation absorbers (sunscreen filters); fragrances; colorants; or any combinations of any of the foregoing.

The above compositions can be formulated as creams, gels, or liquids, and preferably are prepared as lotions. Compositions can be prepared as multi-lamellar vesicles, liposomes, nanospheres, microsponges, or any combination of any of the foregoing by methods known to those skilled in the art.

The present invention also relates to a composition for the cosmetic and/or pharmaceutical treatment of the upper layers of the epidermis, by topical application of the said composition to the skin, and methods for obtaining such a composition.

It is well known in the cosmetics and pharmaceutical arts that an active principle can be administered as an ingredient of oils to be applied to the skin. Such oils are used as they are or, more often, in the form of a water-in-oil or oil-in-water emulsions. Such oils or emulsions containing same are known to exert an action on the surface of the skin, and also in the upper layers of the epidermis, since they can pass through the stratum corneum. In general, the cosmetic and/or pharmaceutical action of the oils generally increases in efficacy with an increase in proportion to the relative quantity of oil penetrating into the upper layers of the epidermis.

The term “nanoparticles” is commonly used to denote colloidal particles of the order of 10 to 1,000 nm in size. Nanoparticles commonly comprise polymeric materials, in which an active principle is trapped, encapsulated and/or adsorbed (see J. KREUTER, J. MICROENCAPSULATION 1988, Vol. 5, pages 115-127). The term nanoparticles can be used to denote nanospheres and nanocapsules: a nanosphere comprises a porous solid polymer matrix on which the active ingredient is adsorbed; a nanocapsule comprises a polymer membrane surrounding a core consisting of the active principle. For the remainder of the description and in the claims, the scope of the term “nanoparticles” shall be intended to denote the nanocapsules as defined above. Among polymers which may be used for the manufacture of nanoparticles, biodegradable materials are usually preferred in order to enable the said nanoparticles to be used therapeutically. It is known that cyanoacrylates, and especially polyalkyl cyanoacrylates, can be used to obtain biodegradable nanoparticles; the preparation of nanoparticles from cyanoacrylates is described in European Patent No. B-0,007,895 and European Patent No. B-0,064,967.

The use of biodegradable nanoparticles encapsulating biologically active compounds has been proposed in many therapeutic applications for many active principles, such as antimitotic or antineoplastic substances, antibiotics, hormonal substances, insulin, heparin or biological products such as proteins, antigens or the constituents of viruses, bacteria or cells. It has hence already been proposed to administer nanoparticles encapsulating active principles orally, subcutaneously, intradermally, intramuscularly, intravenously and by application to the eye (See EP-B-0,007,895, EP-B-0,064,967, FR-B-2,604,903, DE-A-3,722,837, DE-A-3,341,001 and FR-B-2,304,326).

In FR-A-2,515,960, nanoparticles of cyanoacrylate encapsulating an oil or an active substance dispersed in an oil are described, and it is specified that these nanoparticles can be administered orally, subcutaneously, intradermally, intramuscularly or intravenously. In addition, FR-A-2,515,960 also describes the use of nanoparticles for encapsulating perfumes, the encapsulated perfumes allegedly causing the perfume odor to persist longer after application than in the case where the perfume is applied to the skin without encapsulation. In addition, in this case, the desired action of the perfume takes place at the surface of the skin, and the persistence of the odor is completely independent of the fate of the fraction of the nanoparticles which might possibly pass through the stratum corneum. This topical application hence provides no information as to the possible capacity of the nanoparticles to pass through the stratum corneum and to be degraded in the upper layers of the epidermis; instead, it relies upon the prediction that the nanoparticles remain predominantly on the surface of the skin, thus releasing the perfume therein.

The present invention is based on the finding that, by cutaneous topical application of a composition comprising biodegradable nanoparticles encapsulating oils that comprise an active ingredient, an especially effective cosmetic and/or pharmaceutical action is obtained.

Without being bound to any particular theory, it is believed that this action is obtained because the nanoparticles, rather than being all degraded on the epidermis, can pass through the stratum corneum more readily than the unencapsulated oil, regardless of whether the oil is in the form of a water-in-oil or oil-in-water emulsion.

Such an action was unexpected. Although the introduction of nanoparticles into certain types of tissue, especially by injection, has been known to lead to the biodegradation of the nanoparticles, it has also been well known that different tissues have different constitutions and contain different enzymes. In particular, it has been well known that the connective tissue of muscle, dermis and the deep layers of the skin, where nanoparticles have previously been introduced by injection, have a very different biochemical constitution than that of the upper layers of the epidermis (See, for example, British Journal of Dermatology (1976) 94, 443).

The present invention is also based on the finding that the encapsulation of an active oil (or of an oily substance comprising an active ingredient) in nanoparticles produced an immediate action of the composition. This finding was unexpected in view of the delayed action reported for the topical perfume compositions reported in FR-A-2,515,960. Such an immediate action is especially well suited to topical administration, as was shown in a comparative in vitro study of percutaneous absorption.

The compositions of the invention are directed to the cosmetic and/or pharmaceutical treatment of the upper layers of the epidermis, by topical application to the skin, and comprise in a suitable vehicle, biodegradable polymer nanoparticles encapsulating at least one active ingredient. The active ingredient is a compound or composition having cosmetic and/or pharmaceutical action, and is in the form of an oil or comprised in an inactive carrier oil or an active oil.

The nanoparticles of the present invention are preferably between 10 and 1000 nm, and more especially between 50 and 500 nm, in size.

The weight of the nanoparticles loaded with at least one active ingredient advantageously constitutes from 0.1% to 20% of the total weight of the composition, and preferably from 0.5 to 5%. by weight.

The polymers constituting the biodegradable nanoparticles can be polymers of C₂-C₁₂, and especially C₂-C₆, alkyl cyanoacrylate; the alkyl radical is preferably selected from the group composed of ethyl, n-butyl, hexyl, isobutyl and isohexyl radicals. The biodegradable polymers may also be taken from the group composed of poly-L-lactides, poly-DL-lactides, polyglycolides, polycaprolactones, polymers of 3-hydroxybutyric acid and the corresponding copolymers, such as copoly(DL-lactides/glycolides), copoly(glycolides/caprocolatones) and the like.

The use of nanoparticles obtained from poly-L-lactides, poly-DL-lactides and copoly(DL-lactides/glycolides) is especially advantageous, since the products of enzymatic or chemical biodegradation of the nanoparticles can themselves have cosmetic effects: for instance, lactic acid exhibits humectant and plasticising properties; and glycolic acid exhibits depigmenting and/or biostimulatory properties.

The active ingredients in the form of an oil (or active oils) are preferably selected from the group composed of a-tocopherol, a-tocopherol acetate, triglycerides rich in linoleic and/or linolenic acid(s), pentaerythritol tetra(2-ethylhexanoate), clofibrate, tocopherol linoleate, fish oil, hazelnut oil, bisabolol, farnesol, farnesyl acetate, ethyl linoleate and ethylhexyl para-methoxycinnamate.

The inactive carrier oils are preferably selected from the group composed of triglycerides, simple or modified, especially by oxyethylenation, volatile silicone oils and mixtures thereof.

To obtain the loaded nanoparticles used in the composition according to the invention it is possible either to take an active oil, or to introduce into an active oil or into a carrier oil which is in itself inactive, any active ingredient capable of having a cosmetic or therapeutic activity. These active ingredients can be, inter alia, emollients, humectants, free radical-inhibiting agents, anti-inflammatories, vitamins, depigmenting agents, anti-acne agents, antiseborrhoeics, keratolytics, slimming agents, skin coloring agents and sunscreen agents, and in particular linoleic acid, retinol, retinoic acid, ascorbic acid alkyl esters, polyunsaturated fatty acids, nicotinic esters, tocopherol nicotinate, unsaponifiables of rice, soybean or shea, ceramides, hydroxy acids such as glycolic acid, selenium derivatives, antioxidants, β-carotene, γ-orizanol and stearyl glycerate.

The active ingredient is preferably an oleophilic active ingredient in the form of a solution in the oil. However, it can also be in the form of a dispersion, suspension or emulsion.

In the nanoparticles, the weight ratio of the biodegradable polymer of the nanoparticles to the active oily phase is preferably between 0.05 and 0.5, and in particular in the region of 0.2.

The compositions according to the invention can take the form of a physiological fluid, a lotion, an aqueous, aqueous-alcoholic or oily gel or a water-in-oil or oil-in-water emulsion, or alternatively of aqueous dispersions of vesicles in which the constituent lipids are ionic or nonionic lipids or a mixture of ionic and nonionic lipids, with or without an oily phase. Their use to constitute physiological fluids is especially advantageous: in effect, this type of product requires the introduction of a large amount of emulsifier in the case where it is desired to introduce unencapsulated oily active ingredients into them, and it is well known that emulsifiers have the effect of irritating the skin and are not compatible with all active ingredients.

The compositions can contain, in addition to the nanoparticles, known cosmetically and/or pharmaceutically acceptable adjuvants, such as fats, vaseline, preservatives, thickening agents, colorings and perfumes.

When a polymer of (C₂-C₁₂) alkyl cyanoacrylate is used to obtain the nanoparticles of the composition, an interfacial polymerisation of a microemulsion of oil in an aqeuous-alcoholic medium is performed, as described, for example, in FR-A-2,515,960, by injecting, into an aqeuous phase containing or otherwise one surfactant, a mixture consisting of the oil(s) to be encapsulated, at least one (C₂-C₁₂) alkyl cyanoacrylate and at least one solvent which can contain one surfactant, then evaporating off the solvent and optionally concentrating the aqueous dispersion of nanoparticles obtained. The solvent used is, more often than not, a C₂-C₄ lower alcohol, especially ethanol, propanol, isopropanol or a mixture of these alcohols, or alternatively acetone; it can optionally contain one surfactant.

It is also possible to use the process for manufacturing nanoparticles described in European Patent Application No. 0,274,961. In this case, the nanoparticles are obtained by precipitation of the polymer around a dispersion of oily droplets, by injecting, into an aqueous phase containing or otherwise one surfactant, a mixture consisting of the oil(s) to be encapsulated, at least one polymer and at least one solvent containing or otherwise one surfactant, and then evaporating off the solvent.

Other processes for manufacturing nanoparticles may also be used.

The surfactant optionally used in the preparation process can consist of at least one nonionic surfactant, more especially selected from the condensates of glycerol, ethylene oxide and propylene oxide, or of at least one ionic surfactant which can, in particular, be taken from the phospholipid group, such as lecithin, or alternatively of a mixture of at least one surfactant of each of these two categories. This surfactant promotes the formation of the microemulsion of oil, and prevents coalescence of the nanoparticles within the reaction mixture. The weight ratio of the surfactant used on the one hand, to the materials constituting the nanoparticles loaded with active ingredient(s) on the other hand, is advantageously between 0.01 and 0.5, and preferably in the region of 0.2.

When a surfactant used during the process for manufacturing the nanoparticles is in itself capable of forming vesicles consisting of lipid lamellae encapsulating a closed space, the said surfactant behaves in a fundamentally different way according to whether it is introduced into the aqeuous phase or into the solvent phase. If the surfactant is in the aqueous phase, it has a tendency, at least partially, to form vesicles. If, on the other hand, the surfactant is in the solvent phase, it has a tendency, at least partially, to form one or more lipid lamellae, each consisting of a molecular bilayer, around the polymer membrane of each nanoparticle.

In the case where the oil to be encapsulated is a self-emulsifying oil, selected, for example, from oxyethylenated triglycerides, it is not necessary to use a surfactant.

The aqueous dispersion of nanoparticles obtained may be used as it is. It can also be lyophilized, in particular in the presence of anticaking additives such as silicas, sugars, salts, proteins, peptides and amino acids. The lyophilizates have the advantage of enabling anhydrous cosmetic compositions to be prepared. If the nanoparticles are coated with at least one lipid lamella consisting of at least one surfactant capable of forming vesicles, the compositions according to the invention can exhibit especially advantageous cosmetic features. These coated nanoparticles can constitute only a part of the nanoparticles of the composition.

The examples given below, purely by way of illustration and without implied limitation, will facilitate understanding of the invention.

### EXAMPLE 1.

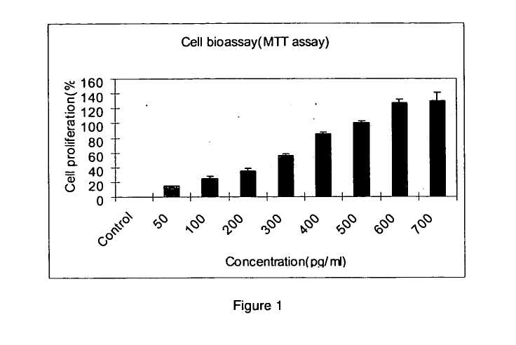

HACAT cells were seeded in 96 well plates and cultured for one day in DMEM medium with 10% FBS, then subjected to starvation for one day. After starvation, the cells were treated for three days with various dosages of EGF in serum free media. As illustrated in FIG. 1, EGF-treated cells showed higher cell proliferation ratios than the untreated controls, as measured by the MTT bioassay. The ED₅₀ was approximately 300 pg/ml.

### EXAMPLE 2.

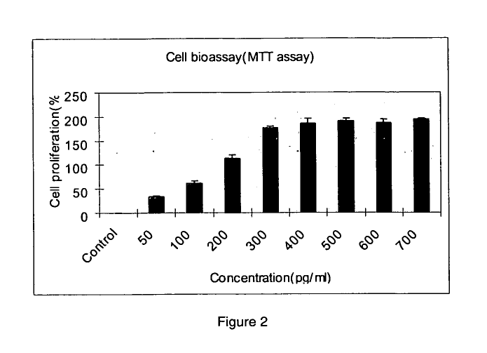

HACAT cells were seeded in 96 well plates and cultured for one day in DMEM medium with 10% FBS, then subjected to starvation for one day. After starvation, the cells were treated for three days with various dosages of Anti-Aging Cytokine Complex AC (“AC ”), i.e. a mixture comprising EGF, bFGF, IGF-1 and TRX, in serum free media. As illustrated in FIG. 2, AC-treated cells showed higher proliferation ratios than the untreated controls, as measured by the MTT bioassay. AC exhibited an ED₅₀ of approximately 90 pg/ml, showing a superior activity than EGF alone.

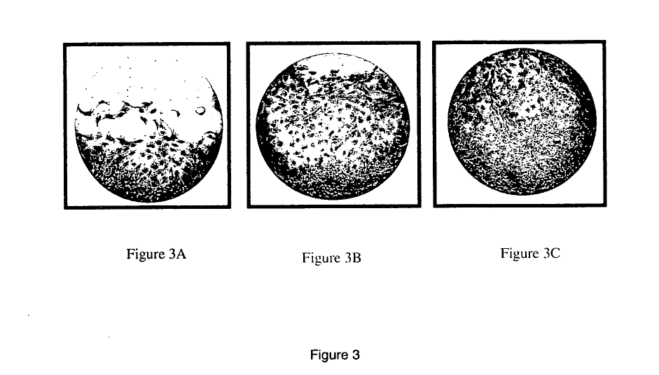

This superior activity is also illustrated in FIG. 3. FIG. 3A depicts control cells, FIG. 3B cells treated with 0.5 ng/ml EGF, and FIG. 3C cells treated with 0.5 ng/ml of AC.

### EXAMPLE 3

Anti-Aging Cytokine Complex(AC) comprising EGF, bFGF, IGF-1 and TRX was formulated in nanocapsules comprising phosphatidyl choline, cholesterol and sodium oleic acid, and the product was formulated in an anti-aging cream for topical application, according to the composition of Table 1, as follows:

TABLE 1

| Ingredient | Quantity(weight %) |
|---|---|
| Anti-Aging Cytokine Complex (comprising | 0.02 |
| 5 ug/g of each of EGF, bFGF, IGF-1 and TRX, | |
| for a total of 20 ug/g) | |
| Petrolatum | 7.0 |
| Liquid paraffin | 10.5 |
| Polysolbate 60 | 2.0 |
| Bees wax | 1.5 |
| Sorbitan sesguioleate | 2.0 |
| Glyceryl stearate | 2.6 |
| Squalene | 3.0 |
| Propylen glycol | 6.0 |
| Glycerin | 4.0 |
| Triethanol amine | 0.5 |
| Carboxyvinyl polymer | 0.5 |
| Tocopherol acetate | 0.1 |
| Methylparaben | 0.2 |
| Fragrance | q.s. |
| Distilled water | 60.08 |
| Total | 100 |

### EXAMPLE 4

The AC-containing cream of Example 3 was applied twice a day with dose of 0.2 g for 6 weeks on face of groups comprising twenty female subjects aged 30 years or older. Then, replicas of the skin wrinkles of the treated subjects and of an untreated control group were prepared using transparent silicon solution. The changes in the skin wrinkles of the replicas were detected with a Skin Visiometer SV400® (Courage-Khazaka Electronics GmbH, Germany). Then three-dimensional images of the replicas were analyzed with a CCD camera. The skin wrinkle improvement effect was determined as average roughness of the wrinkles (R₂) according to the following numerical formula

TABLE 2
${R}_{z} = \frac{{R}_{1} + {R}_{2} + {R}_{3} + {R}_{4} + … + {R}_{m - 1} + {R}_{m}}{Number of wrinkles ( m )}$

ΔR_{z} = Initial R_{z}—R_{z} determined at week 6

| | Control Group | Treated Group |
|---|---|---|
| Subject 1 | 0.010 | 0.070 |
| Subject 2 | 0.021 | 0.075 |
| Subject 3 | 0.012 | 0.084 |
| Subject 4 | 0.015 | 0.067 |
| Subject 5 | 0.021 | 0.073 |
| Subject 6 | 0.010 | 0.079 |
| Subject 7 | 0.003 | 0.057 |
| Subject 8 | 0.008 | 0.064 |
| Subject 9 | 0.011 | 0.090 |
| Subject 10 | 0.031 | 0.088 |

Table 2 reports example results obtained in ten treated subjects and ten control subjects. In case of the treated group, the height (roughness) of the wrinkles was decreased by 0.06-0.09 mm. No decrease in skin wrinkle height was detected in the control group.

### EXAMPLE 5

Sixty female subjects aged 30 years or older were divided into three groups ( Group 1, Group 2, Control Group). A cream was applied twice daily to the face of each subject, with a dosage of 0.2 g per application, for a duration of six weeks. The subjects were divided into three groups of twenty subjects each. Any change in skin wrinkles on both sides of the face was measured twice daily for three months after the pre-measurement of the same skin sites.

Skin evaluation was carried out at 24 Celsius degrees, 40% relative humidity in an air-conditioned room. Replicas of the skin wrinkles were then prepared with a transparent silicon solution. The replicas were taken from the crow's feet area and the changes in the skin wrinkles of the replicas were detected with Skin Visiometer SV400®. The images of the replicas were analyzed three dimensionally with a video-sensor charge coupled device (CCD) camera and the skin wrinkles were analyzed in terms of average decrease of wrinkle height (roughness) three months after the beginning of the test, as described in Table 3.

TABLE 3

| | Group 1 | Group 2 | Control Group |
|---|---|---|---|
| Subjects | (mm) | (mm) | (mm) |
| Subject 1 | 0.140 | 0.142 | 0.045 |
| Subject 2 | 0.143 | 0.163 | 0.051 |
| Subject 3 | 0.133 | 0.137 | 0.044 |
| Subject 4 | 0.132 | 0.155 | 0.040 |
| Subject 5 | 0.137 | 0.152 | 0.038 |
| Subject 6 | 0.129 | 0.143 | 0.078 |
| Subject 7 | 0.147 | 0.147 | 0.062 |
| Subject 8 | 0.141 | 0.148 | 0.050 |
| Subject 9 | 0.144 | 0.139 | 0.043 |
| Subject 10 | 0.135 | 0.142 | 0.050 |
| Subject 11 | 0.152 | 0.138 | 0.041 |
| Subject 12 | 0.151 | 0.144 | 0.053 |
| Subject 13 | 0.149 | 0.153 | 0.043 |
| Subject 14 | 0.135 | 0.145 | 0.046 |
| Subject 15 | 0.137 | 0.139 | 0.047 |
| Subject 16 | 0.153 | 0.140 | 0.041 |
| Subject 17 | 0.134 | 0.133 | 0.063 |
| Subject 18 | 0.135 | 0.145 | 0.052 |
| Subject 19 | 0.121 | 0.143 | 0.057 |
| Subject 20 | 0.138 | 0.148 | 0.051 |
| Average | 0.139 | 0.145 | 0.050 |

In case of the Control Group, the measured decrease in wrinkle height was between 0.03 and 0.078 mm, with an average of 0.05 mm. In Group 1, the measured decrease in wrinkle height was between 0.121 mm and 0.153 mm, with an average of 0.139 mm, thus indicating wrinkle improvement within the range of statistical significance (P<0.01). In Group 2, the measured wrinkle height decrease was between 0.133 and 0.163 mm, with an average of 0.145 mm, also indicating wrinkle improvement within the range of statistical significance (P<0.01).

### EXAMPLE 6

Glycoprotein of Ulmus davidiana Nakai (UDN glycoprotein) was isolated and identified using SDS-PAGE. UDN glycoprotein was shown to have strong scavenging activities against oxygen free radicals, as detected by different oxygen-radical formation assays. To investigate the anti-apoptotic effects of UDN glycoprotein, we investigated the activity of protein kinase C alpha (PKCalpha), the DNA-binding activation of nuclear factor-kappa B (NF-kappaB), the production of nitric oxide (NO) and apoptosis in 12-Otetradecanoylphorbol 13-acetate (TPA)-stimulated NIH/3T3 cells using a western blot analysis, electrophoretic mobility shift assays (EMSA) and NO assays. Results in this experiment showed that 100 microg/ml of UDN glycoprotein has inhibitory effects on PKCalpha translocation, NF-kappaB DNA binding activity, NO production, and apoptosis in TPA (61.68 ng/ml)-stimulated NIH/3T3 cells. Interestingly however, it could not regulate the DNA binding activity of AP-1. Therefore, UDN glycoprotein, a natural anti-oxidant, is a potential modulator of apoptotic signal pathways in NIH/3T3 cells.

### OTHER EMBODIMENTS

The present invention also provides methods and compositions for treating hair loss, for instance treatments with bFGF, IGF-1 and VEGF. Also provided are methods and compositions for treating acne, for example treatments with EGF, IGF-1, IGY, Nicin. In addition, methods and compositions for treating psoriasis, for example treatments with IL-10, are provided.

Example methods and treatments for anti-atopic dermatitis according to the invention comprise, for example, treatments with IL-10 and TRX. In a further set of embodiments, the present invention provides sun-block methods and compositions, for instance by treatment with EGF and TRX.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

### REFERENCES CITED

Throughout this application various publications have been referenced. The disclosures of these publications in their entireties are hereby incorporated by reference in this application in order to more fully describe the state of the art to which this invention pertains.

References:

Pretolani M. Interleukin-10: an anti-inflammatory cytokine with therapeutic potential.
Clin. Exp. Allergy 29:1164-1171 (1999).
Sahni A., et al. (2003) J. Thromb. Haemost. 1:1304-1310.
Bond J. J., et al. (1998) Acta Derm. Venerol. 78:337-342.
Igarashi M., et al. (1998) J. Biol. Chem. 273: 13230-13235.
Soler P. M., et al. (1999) Wound Repair Regen. 7:172-178.
Woelfle J., et al. (2005) Pediatr. Nephrol. 20:295-302.
Lee S. J., et al. (2004) Toxicol Lett. Jan 15;146(2):159-74

What is claimed:

1. A method of treatment for slowing the progress of skin aging comprising contacting the skin with an amount effective to slow skin aging of a composition comprising one or more compounds selected from the group consisting of EGF, bFGF, IGF-1, KGF, TGF-β3, TRX-1, VEGF, aFGF, FGF-10, copper peptide, acetyl hexapeptide, palmitoyl pentapeptide, CPP, and UDN glycoprotein.

2. The method of claim 1 wherein the compounds are each at a concentration of 5 μg/g.

3. The method of claim 1 wherein the compounds each have a concentration of approximately 0.00001% to 0.01%.

4. The method of claim 1 wherein the composition further comprises microparticles.

5. The method of claim 1 wherein the composition comprises nanoparticles.

6. The method of claim 1 wherein the composition comprises EFG, bFGF, IGF-1 and TRX.

7. The method of claim 6 wherein the EFG, bFGF, IGF-1 and TRX are each at a concentration of 5 μg/g.

8. The method of claim 6 wherein the EFG, bFGF, IGF-1 and TRX each have a concentration of approximately 0.00001% to 0.01%.

9. The method of claim 6, wherein the composition further comprises microparticles.

10. The method of claim 6, wherein the composition further comprises nanoparticles.

11. A composition for slowing the progress of skin aging, the composition comprising one or more compounds selected from the group consisting of EGF, bFGF, IGF-1, KGF, TGF-μ3, TRX, VEGF, aFGF, FGF-10, copper peptide, acetyl hexapeptide, palmitoyl pentapeptide, CPP, and UDN glycoprotein.

12. The composition of claim 11 wherein the EFG, bFGF, IGF-1 and TRX are each at a concentration of 5 μg/g.

13. The composition of claim 11 further comprising microparticles.

14. The composition of claim 11 further comprising nanoparticles.

15. The composition of claim 11 comprising EFG, bFGF, IGF-1 and TRX.

16. The composition of claim 15 wherein the EFG, bFGF, IGF-1 and TRX are each at a concentration of 5 μg/g.

17. The composition of claim 15 wherein the EFG, bFGF, IGF-1 and TRX each have a concentration of approximately 0.00001% to 0.01%.

18. The composition of claim 15 further comprising microparticles.

19. The composition of claim 15 further comprising nanoparticles.

20. A method of treating hair loss comprising contacting the skin with an amount effective to treat hair loss of a composition comprising one or more compounds selected from the group consisting of bFGF, KGF, IGF-1, SCF, VEGF, copper peptide, aFGF, Noggin and thymosinβ4.

21. The method of claim 20, wherein the composition comprises bFGF, KGF, IGF-1, SCF, VEGF, copper peptide, aFGF, Noggin and thymosinβ4.

22. A composition for treating hair loss, the composition comprising one or more compounds selected from the group consisting of bFGF, IGF-1, and VEGF.

23. A method for treating acne comprising contacting the skin with a composition comprising one or more compounds selected from the group consisting of EGF, bFGF, TRX, UDN glycoprotein, IL-4, IL-10 and SCF.

24. A composition for treating acne, the composition comprising one or more compounds selected from the group consisting of EGF, bFGF, TRX, UDN glycoprotein, IL-4, IL-10 and SCF.

25. A method for treating acne comprising contacting the skin with a composition comprising one or more compounds selected from the group consisting of EGF, IGF-1, bFGF, copper peptide and UDN glycoprotein.

26. A composition for treating acne, the composition comprising one or more compounds selected from the group consisting of EGF, IGF-1, bFGF, copper peptide and UDN glycoprotein.

27. A method for treating atopic dermatitis comprising contacting the skin with a composition comprising one or more compounds selected from the group consisting of EGF, bFGF, TRX, UDN glycoprotein, IL-4, IL-10 and SCF.

28. A composition for treating atopic dermatitis, the composition comprising one or more compounds selected from the group consisting of EGF, bFGF, TRX, UDN glycoprotein, IL-4, IL-10 and SCF.

29. A method for treating psoriasis comprising contacting the skin with an amount effective to treat psoriasis of a composition comprising one or more compounds selected from the group consisting of EGF, bFGF, TRX, UDN glycoprotein, IL-4, IL-10 and SCF.

30. The method of claim 29 wherein the composition comprises IL-10.

31. A composition for treating psoriasis, the composition comprising one or more compounds selected from the group consisting of EGF, bFGF, TRX, UDN glycoprotein, IL-4, IL-10 and SCF.

32. The composition of claim 31 wherein the composition comprises IL-10.

33. A method for increasing lipogenesis comprising administering to a patient an amount effective to increase lipolysis of a composition comprising IGF-1, and L-carnitine.

34. The method of claim 33 wherein the composition further comprises caffeine.

35. A composition for increasing lipogenesis, the composition comprising IGF-1, and L-carnitine.

36. The composition of claim 35 further comprising caffeine.

37. A method for treating leukoplakia comprising contacting the skin with an amount effective to treat leukoplakia of a composition comprising one or more compounds selected from the group consisting of bFGF and SCF.

38. A composition for treating leukoplakia, the composition comprising one or more compounds selected from the group consisting of bFGF and SCF.

39. A method for reducing sun exposure of the skin comprising applying to the skin a composition comprising one or more compounds selected from the group consisting of EGF, bFGF, IGF-1, TRX, CPP, and UDN glycoprotein.

40. The method of claim 39 wherein the composition comprises EGF and TRX.

41. A composition for reducing sun exposure of the skin, the composition comprising one or more compounds selected from the group consisting of EGF, bFGF, IGF-1, TRX, CPP, and UDN glycoprotein.

42. The composition of claim 41 wherein the composition comprises EGF and TRX.

43. A method for treating atopic dermatitis comprising contacting the skin with an amount effective to treat atopic dermatitis of a composition comprising one or more compounds selected from the group consisting of IL-10, TRX, EGF and bFGF.

44. The method of claim 43 wherein the composition comprises IL-10 and TRX.

45. A composition for treating atopic dermatitis, the composition comprising one or more compounds selected from the group consisting of IL-10, RRX, EGF and bFGF.

46. The composition of claim 45 wherein the composition comprises IL-10 and TRX.
